(19) United States
(12) Patent Application Publication
(10) Pub. No.: US 2018/0133194 A1
(43) Pub. Date: May 17, 2018

(54) METHOD OF TREATING INFLAMMATION USING NATURAL COMPOUNDS AND/OR DIET

(71) Applicant: UNIVERSITY OF FLORIDA RESEARCH FOUNDATION, INCORPORATED, GAINESVILLE, FL (US)

(72) Inventors: LOIC PIERRE DELEYROLLE, GAINESVILLE, FL (US); BRENT ALLAN REYNOLDS, GAINESVILLE, FL (US)

(21) Appl. No.: 15/578,601

(22) PCT Filed: Jun. 27, 2016
(86) PCT No.: PCT/US2016/039534

§ 371 Date: Nov. 30, 2017

Related U.S. Application Data
(60) Provisional application No. 62/185,001, filed on Jun. 26, 2015.

(51) Int. Cl. A61K 31/353; A61K 31/12; A61K 31/26; A61K 31/19; A61K 31/20; A61P 37/08; A61P 25/28; A61P 19/02; A61P 11/06; A61P 9/10; A61P 25/00; A61P 1/00; A61P 17/00; A61P 1/18; A61P 25/16; A61P 17/06; A61P 37/02; A61P 5/00; A61P 17/14; A61P 13/12; A61P 31/04

(52) U.S. Cl. CPC A61K 31/353; A61P 31/04; A61K 31/26; A61K 31/19; A61K 31/20; A61P 37/08; A61P 25/28; A61P 19/02; A61P 11/06; A61P 9/10; A61P 25/00; A61P 1/00; A61P 17/00; A61P 1/18; A61P 25/16; A61P 17/06; A61P 37/02; A61P 5/00; A61P 17/14; A61P 13/12; A61K 31/12

(57) ABSTRACT

The current invention is directed to a treatment of an inflammatory disease comprising administering to a subject in need of such treatment a composition comprising epigallocatechin-3-gallate (EGCG), curcumin, glucosinolates and/or derivatives thereof and medium chain triglycerides and, optionally, providing a ketogenic diet or a modified ketogenic diet to the subject. In an embodiment, the inflammatory disease is increased inflammation in the subject caused by the administration of a microtubule stabilizing drug such as paclitaxel.

### CROSS-REFERENCE TO RELATED APPLICATION

This application claims the benefit of U.S. Provisional Application Ser. No. 62/185,001, filed Jun. 26, 2015, the disclosure of which is hereby incorporated by reference in its entirety, including all figures, tables and amino acid or nucleic acid sequences.

### BACKGROUND OF THE INVENTION

Inflammatory diseases are a major health concern. The currently available treatments involve small molecule compounds or antibodies directed to various agents involved in inflammatory pathways. The current treatments against inflammatory diseases cause substantial side effects.

### BRIEF SUMMARY OF THE INVENTION

The invention provides methods of reducing inflammation and treating inflammatory diseases which do not exhibit undesirable side effects. In one embodiment, the invention provides a method of treating an inflammatory disease, the method comprising administering to a subject in need of a treatment against the inflammatory disease a composition comprising one or more natural products (compounds) and, optionally, simultaneously providing to the subject a low-carbohydrate diet. In certain embodiments of the invention, the low-carbohydrate diet is a ketogenic diet (KD), a modified ketogenic diet (mKD) or an Atkins-like diet. Thus, the method of treating an inflammatory disease comprises administering to a subject a composition comprising one or more compounds (component(s)) selected from epigallocatechin-3-gallate (EGCG), curcumin, glucosinolates and/or derivatives thereof (such as glucoraphanin (GRP) and/or sulforaphane (SFN) as found in broccoli sprouts or sprouts of other cruciferous vegetables), and medium chain triglycerides (MCT) and, optionally, providing a low carbohydrate diet such as an Atkins diet, mKD or KD to the subject. The subject application provides a variety of natural compounds alone or in combination with MCT, KD, mKD, and ketones for the treatment of inflammatory diseases. These combination treatments are referred to as: NU.001=[EGCG+curcumin+glucosinolates]+[KD or mKD]+MCT; NU.002=[EGCG+curcumin+glucosinolates]; NU.003=[EGCG+curcumin+glucosinolates]+MCT; NU.004=[EGCG+curcumin+glucosinolates]+Ketones; and NU.005=mKD+MCT.

### BRIEF DESCRIPTION OF DRAWINGS

FIG. 1. Using a drug induced model of inflammation, a cytokine array was used to measure inflammatory cytokines, immune system alteration and the ability of our treatment to attenuate and re-establish normal status. Plasma was isolated from mice treated with the different treatments (control, paclitaxel [PTX, 40 mg/kg], NU.001[CS] and PTX+NU.001[PTX+CS]). * indicates p<0.05 and ** indicates p<0.01, one-way ANOVA or t-test, compared to control. # indicates p<0.05 and ## indicates p<0.01, one-way ANOVA or t-test, compared to PTX.

FIG. 2. NU.001 reduces inflammatory signals in a drug-induced model of inflammation. Animals received oral delivery of NU.001 for 3-4 weeks or a control diet. The microtubule stabilizing agent paclitaxel [40 mg/kg cumulative] was administered to induce inflammation. A cytokine array was used to measure inflammation and immune system status and assess the ability of NU.001 to attenuate or re-establish normal cytokine levels. Plasma was isolated from mice treated with the different treatments (control, paclitaxel, NU.001, and paclitaxel+NU.001). The results show that inflammation related cytokines are up-regulated under the influence of paclitaxel and that introducing NU.001 is able to re-establish levels of lymphotactin, thrombopoietin, vascular endothelial growth factor A [VEGF-A], and interleukin 18 [IL-18] similar to control levels. *, p<0.05, one-way ANOVA, compared to control. Treatment composition is as follows: [1] Control=55% carbohydrates, 30% proteins, 15% fat, [2] NU.001=10-20% carbohydrates, 50-60% fat (about half coming from MCT), 30% proteins+curcumin [1200 mg/kg of body weight], EGCG [1200 mg/kg of body weight]), SFN [25 mg/kg of body weight].

FIG. 3. NU.001 is able to reduce pro-inflammatory effectors. After being fed for 3-4 weeks with control diet or NU.001 diet, mice underwent blood draw for subsequent plasma isolation. A cytokine array was used to assess inflammation status. The results demonstrate the ability of NU.001 to reduce pro-inflammatory effectors such as tissue inhibitor of metalloproteinase-1 [TIMP1], macrophage inflammatory protein-1 gamma [MIP1-g], leptin, macrophage colony-stimulating factor [MCSF], and keratinocyte-derived cytokine/growth related protein [KC/GRO]. *, p<0.05, t-test.

FIGS. 4A-4B. NU.001 increases levels of Leukemia inhibitory factor [LIF] and CCL22. A. After being fed for 3-4 weeks with control diet or NU.001 diet, plasma was isolated for cytokine screening. FIG. 4A indicates the ability of NU.001 to stimulate the expression of LIF. LIF has been proposed to prevent or treat peripheral neuropathy. *, p <0.05, t-test. B. Paclitaxel treatment induced a decrease of macrophage-derived chemokine [MDC/CCL22], a cytokine that has been described to be down-regulated in patients diagnosed with multiple sclerosis. The presented graph confirms NU.001 as an immunomodulator with ability to mitigate, prevent or delay significantly MDC/CCL22 deficit. **, p<0.01, one-way ANOVA, compared to control, #, p<0.05, t-test, compared to paclitaxel.

### DETAILED DESCRIPTION OF THE INVENTION

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed. In the context of compositions containing amounts of ingredients where the terms “about” or “approximately” are used, these compositions contain the stated amount of the ingredient with a variation (error range) of 0-20% around the value (X±20%). When ranges are used herein, such as for dose ranges, combinations and subcombinations of ranges (e.g., subranges within the disclosed range) and specific embodiments therein are intended to be explicitly included.

The terms “treatment”, “treating”, “palliating” and “ameliorating” (and any grammatical variation of these terms) may be used interchangeably. These terms refer to an approach for obtaining beneficial or desired results including but not limited to therapeutic benefit. A therapeutic benefit is achieved with the eradication or amelioration (lessening) of one or more of the physiological symptoms associated with the underlying disorder such that an improvement is observed in the patient, notwithstanding that the patient may still be afflicted with the underlying disorder. In the context of this invention, amelioration of symptoms includes returning (reducing) measured cytokine levels to non-inflammatory (normal) levels in the subject. In certain embodiments, measured cytokine levels are reduced by at least 25%, 30%, 35%, 40%, 45%, 50%, 55%, 60%, 65%, 70%, 75%, 80%, 85%, 90%, 95% or more. Similarily, in the context of this invention, amelioration of symptoms includes increasing cytokine levels that may be reduced due to treatment, injury, disease, physiological imbalance or disharmony. In the context of this invention, amelioration of physiological symptoms due to treatment, disease, injury, physiological balance or disharmony can accomplished by rasing normal cytokine levels.

The invention provides that administering certain natural compounds to a subject, optionally, in combination with mKD or KD, changes inflammatory cytokines that are well-established as inflammation promoters. For example, animals treated with paclitaxel exhibited increased cytokines and related inflammatory molecules. Such animals, when treated with the combination of natural products provided by the invention, optionally, in combination with mKD or KD, exhibited the return of cytokines in the blood to control levels, where the expression and/or amount of the cytokines was altered as a result of the paclitaxel treatment.

Thus, one embodiment of the invention provides a method of treating an inflammatory disease in a subject, the method comprising administering to the subject, a composition comprising one or more component(s) selected from: EGCG; curcumin; glucosinolates and/or derivatives thereof and MCT, either alone or in combination with a low carbohydrate diet, for example, KD or mKD. The components can be administered to the subject individually or in various combinations (e.g., pairs, three-component compositions or a single composition containing all components). Another embodiment of the invention provides for treating a subject with a combination treatment corresponding to NU.001; NU.002; NU.003; NU.004 or NU.005 in order to reduce inflammatory disease in the subject.

EGCG is the most abundant catechin in green tea. Polyphenols derived from green tea are well-known to have anti-inflammatory, antioxidant properties and have been demonstrated to play a role in inhibiting tumor cell proliferation in multiple animal models of cancer. These actions are seen at micromolar concentrations that can be achieved by oral ingestion of EGCG.

EGCG: Protective Effect and Inflammation

EGCG has demonstrated anti-inflammatory effects and protective effects in many settings and cell types. For instance, EGCG protects neurons from a variety of toxic agents. It directly functions as a reactive oxygen species (ROS) scavenger and activates antioxidant enzymes. EGCG additionally decreased activation of neuronal apoptosis and reduced activating inflammatory signals to microglial cells. EGCG activates Protein Kinase C gamma signaling which reduces apoptotic signals and protects against cytoskeletal degradation. Additionally EGCG appears to stimulate neurite outgrowth which may promote the regain of lost neurologic function. EGCG is currently in clinical trials for neuroprotective effects in Alzheimer's disease, multiple sclerosis, diabetes, and Parkinson's disease.

Safety of EGCG

Oral doses as high as 500 mg/kg in rodents were found to have no genotoxic or short term toxicity, a dosage that is significantly higher than that proposed for humans. Similarly, no adverse events or toxicity was seen when 500 mg/kg/day was delivered to pre-fed dogs in a divided dosage for 13 weeks. Epidemiological data indicates that nearly a quarter of Japanese consume more than 10 cups of green tea a day, which is the equivalent of approximately 1000 mg of EGCG daily. The amounts of EGCG that can be administered in accordance with the claimed invention range from about 1 mg/kg to about 500 mg/kg. In certain embodiments, EGCG is administered in amounts ranging from about 1 mg/kg to about 250 mg/kg, about 5 mg/kg to about 50 mg/kg, or about 10 mg/kg to about 25 mg/kg. A preferred human dosage of about 1 mg/kg to about 20 mg/kg.

Curcumin is the active component of the dietary spice turmeric. The biological functions of curcumin are diverse and range from anti-tumor, anti-oxidative, anti-viral, anti-amyloid, anti-bacterial and anti-hepatotoxic activities. Curcumin has been evaluated using many neuropathy models and specifically decreased oxaliplatin-induced demyelination and prevented cisplatin-mediated suppression of neurite outgrowth without diminishing anticancer effects. Curcumin has demonstrated reduction of neuropathic pain in clinical trials of patients with sciatica and carpal tunnel syndrome. It has been demonstrated to alleviate neuropathic pain via actions on the monoamine system and reduce diabetic neuropathy through reduction of oxidative stress and inhibition of NF-kappa b activation of TNF-alpha and IL-6 in animal models. This anti-inflammatory effect is also observed in ischemia models to be mediated through NF-κB signaling. Currently, the effect of curcumin on neuropathology is in human trials for Alzheimer's, optic neuropathy, and spinal cord injury.

Safety of Curcumin

The average consumption of curcumin in the typical Indian diet is about 100 mg a day. Several toxicity studies in animals at high doses have shown it to be safe in preclinical models such as rats, guinea pigs and monkeys. Clinical studies have shown the safety of curcumin up to 8000 mg/day at doses of for up to 3 months. Doses escalating from 5000 to 12,000 mg/day showed no significant adverse side effects. Several clinical studies (mostly single-arm phase II) have indicated the effectiveness of curcumin in chronic inflammation, pre-malignant and malignant lesions and AIDS. The amounts of curcumin that can be administered in accordance with the claimed invention range (as daily doses) from about 1 mg to about 12000 mg. In certain embodiments, curcumin is administered in amounts ranging from about 1 mg to about 8000 mg, 5000 mg to about 12000 mg, or about 1000 mg/kg to about 10000 mg. A preferred human dosage of about 1 mg/kg to about 200 mg/kg.

Cruciferous vegetables contain isothiocyanates (ITC) which are formed by hydrolysis of their precursor parent molecule glucosinolates. One of the most studied cruciferous vegetable ITCs is SFN whose precursor Glucoraphanin [GRP] is abundant in broccoli, cauliflower and cabbage, with the highest concentration being found in broccoli sprouts. Hydrolysis of GRP requires the activity of myrosinase enzymes that are present in the vegetables themselves and in the microflora of the colon. SFN is rapidly absorbed with 80% bioavailability, attains peak plasma levels within 2 hours and is characterized by a long terminal elimination phase. Importantly, SFN is a potent inhibitor of Phase I enzymes and stimulator of Phase II enzymes [via NrF2], and can reduce oxidative stress and inhibit NF-kB. In addition, SFN is a potent HDAC inhibitor.

SFN: Protective Effects and Inflammation

SFN, like other isothiocyanates, has been shown to raise tissue glutathione levels, augmenting the cellular antioxidant defenses inherent within virtually all cells. Additional animal and human studies have shown induction of numerous Phase II enzymes (via the Nrf2 pathway mentioned above), including superoxide dismutase, catalase, NAD(P)H:quinine oxidoreductase 1, glutathione peroxidase, glutathione reductase and glutathione-s-transferase. A randomized, double-blind clinical trial also demonstrated SFN's ability to reduce oxidative stress in type 2 diabetes. SFN has been shown to protect neural mitochondria by activating Nrf2 and reduce neuroinflammation by inhibiting NF-KB. Furthermore, SFN has been studied mostly for its anti-carcinogenic effects, and its antioxidative and neuroprotective effects against hypoxic-ischemic injury in a neonatal rat model was also studied. It was observed that SFN treatment increased the expression of Nrf2 antioxidative transcription factor in the brain. SFN also reduced infarct ratio at 24 hours after hypoxic ischemia, and significantly decreased the number of apoptotic cells.

Safety of SFN

Broccoli sprouts are widely consumed as a food all over the world, without any reported adverse effects. Research studies performed in humans have not demonstrated any significant adverse effects of administration of SFN or SFN-enriched dietary origin items such as broccoli sprouts. Increasing evidence supports the view that SFN is considered to be of low toxicity.

An oral intake of 68 grams of broccoli sprouts is demonstrated to provide a safe non-toxic dose [100 mg] of SFN that has proven therapeutic in cancer models. 81 patients with type 2 diabetes were treated for 4 weeks with a dose of up to 10 grams of broccoli sprout powder with no reported side effects. The amounts of glucoraphanin or derivatives thereof, such as SFN, that can be administered in accordance with the claimed invention range (as daily doses) from about 1 mg to about 1000 mg. In certain embodiments, glucoraphanin or derivatives thereof, such as SFN, is administered in amounts ranging from about 1 mg to about 800 mg, 50 mg to about 120 mg, or about 10 mg/kg to about 250 mg. A preferred human dosage of about 0.1 mg/kg to about 5 mg/kg.

Calciferous vegetables contain a group of substances known as glucosinolates, which are sulfur-containing chemicals. During digestion, food preparation or chewing, the glucosinolates are broken down into a number of biologically active compounds, including, but not limited to: indoles, nitriles, thiocyanates, isothiocyanates, Indole-3-carbinol and SFN.

SFN is a bioactive molecule derived from the conversion of a glucosinolate precursor, GRP, found in cruciferous vegetables (for example, Brussels sprouts, cabbage, cauliflower, bok choy, kale, collards, Chinese broccoli, broccoli raab, kohlrabi, mustard, turnip, radish, arugula, and watercress). The highest concentration of SFN is found in broccoli sprouts. Effective doses of glucosinolates, such as GRP and its biologically active breakdown products including SFN, can be delivered by consumption of sprouts or sprout powders derived from the aforementioned cruciferous vegetables or plants from the genus Brassica.

The phrases “composition(s) comprising glucosinolates and/or derivatives thereof, such as GRP and/or SFN” or “composition(s) comprising glucosinolates” or “composition(s) comprising GRP” or “composition(s) comprising SFN” may comprise one or more powders of mature plants of the genus Brassica or mature cruciferous vegetables, consumable vegetative matter of mature plants of the genus Brassica or mature cruciferous vegetables, dehydrated or non-dehydrated sprouts of plants of the genus Brassica or sprouts of cruciferous vegetables, or powdered sprouts obtained from cruciferous vegetables or from plants of the genus Brassica.

In some embodiments, the composition(s) comprising glucosinolates and/or derivatives thereof comprise powders of mature plants of the genus Brassica or mature cruciferous vegetables, consumable vegetative matter of mature plants of the genus Brassica or mature cruciferous vegetables, powders formed from dehydrated or non-dehydrated sprouts of plants of the genus Brassica or sprouts of cruciferous vegetables, or powdered sprouts obtained from cruciferous vegetables or from plants of the genus Brassica. As discussed above, powders from one or more cruciferous vegetable or plants from the genus Brassica can be combined into a composition comprising glucosinolates and/or derivatives thereof. The powders discussed above may be provided in the form of freeze-dried powders. The administration of such powders delivers glucosinolates, including GRP, a compound subsequently metabolized to SFN by myrosinase, to the subject being treated.

The KD diet stimulates the hepatic ketogenic pathway of metabolism. KD may be a potential treatment for a number of neurological disorders and its broad neuroprotective properties may be mediated by altering cellular metabolism, allowing neural cells to resist metabolic changes and upregulate protective mechanisms via antioxidant and anti-inflammatory mechanisms. KD has high fat [90% of caloric intake] and very low carbohydrate [less than 5%] which results in an increase in serum ketone bodies, and reduction in glucose levels that mimicks the effects of fasting or starvation. Several variations of KD are available, such as the modified Atkins diet and the MCT diet, which are aimed at easing the severe carbohydrate restriction and excessive fat consumption posed by the traditional ketogenic diet and increasing compliance by making the diet more palatable and healthy.

As such, the KD is a diet wherein the carbohydrate content is less than, or equal to, about 5% of the total caloric intake of the subject each day and the balance of the diet consists of fats or proteins. Thus, the diet provides, as a function of total caloric intake each day, about 5% or less carbohydrate, about 30% to about 90% fat and about 5% to about 70% protein. In certain embodiments, the diet provides about 3% (or less) carbohydrate, about 57% to about 95% fat, and about 5% to about 40% protein. In some embodiments, from about 30% to about 70% (e.g., about 30%, about 40%, about 50%, about 60% or about 70%) of the fat content of the subject's diet can be made up of MCT. Other embodiments provide that MCT make up about 50% of the fat content of the subject's diet.

Development of mKD

While the KD may have applications for modulating inflammation, it is difficult to implement due to its stringent nature (90-95% fat). The two key physiological changes that occur when on a ketogenic diet are a lowering of glucose levels and an elevation of circulating ketones. mKD mimics the key physiological effects of KD. mKD involves consuming a low-carbohydrate diet [10-20% range] so as to reduce glucose levels and consuming MCT, which elevates blood ketone levels.

The mKD is a diet that contains at least 5% and no more than about 20% carbohydrates (as a function of total caloric intake by the subject each day) and the balance of the diet for the subject comprises fats and proteins. Thus, the diet can, as a function of total caloric intake each day, contain about 5% to about 20% carbohydrates, about 30% to about 75% fats and about 5% to about 65% proteins. In certain embodiments, the diet can provide between about 8% and about 15% carbohydrates, about 50% to about 70% fats and about 18% to about 42% proteins. In some embodiments, from about 30% to about 70% (e.g., about 30%, about 40%, about 50%, about 60% or about 70%) of the fat content of the subject's diet can be made up of MCT. Other embodiments provide that MCT make up about 50% of the fat content of the subject's diet.

As a function of the total amount of food (grams) based on a daily intake of 2000 kilocalories (and based on the fact that 1 g of carbohydrates provides 4 kilocalories, 1 g of fat provides 9 kilocalories, 1 g of proteins provides 4 kilocalories and 1 g of MCTs provide 6.8 kilocalories) the modified ketogenic diet is a diet that contains at least 25 g and no more than 100 g of carbohydrates and the balance of the diet for the subject comprises fats and proteins. Thus, the diet can, as a function of total grams of intake each day, contain about 25 g to 100 g of carbohydrates, about 67 g to about 167 g of fats and about 25 g to about 325 g of proteins. In certain embodiments, the diet can provides between about 40 g and about 75 g of carbohydrates, about 111 g to about 155 g of fats and about 90 g to about 210 g of proteins. In some embodiments, from about 30% to about 70% (e.g., about 30%, about 40%, about 50%, about 60% or about 70%) of the fat content of the subject's diet can be made up of medium chain triglycerides (MCT). This represents from about 40 g to about 165 g of MCTs.

In another embodiment of the invention, the treatment comprises providing, to a subject in need of a treatment for an inflammatory disease, an mKD or KD diet and, optionally, administering a composition comprising one or more of EGCG, curcumin, glucosinolates and/or derivative thereof and MCT. Various embodiments provide for the administration of a composition comprising EGCG, curcumin, glucosinolates and/or derivatives thereof and MCT to the subject.

In one embodiment of the invention, the composition comprising one or more of EGCG, curcumin, glucosinolates and/or derivatives thereof such as GRP or SFN and MCT where at least one of these compounds are present naturally. Furthermore, compositions administered to a subject can be administered as a single combination (e.g., each of EGCG, curcumin, compositions comprising glucosinolates and/or derivatives thereof, such as GRP or SFN, and/or MCT in a single composition) or each of the components (EGCG, curcumin, compositions comprising glucosinolates and/or derivatives thereof such as GRP or SFN, and MCT) can be provided separately for simultaneous or sequential consumption (e.g., in the form of capsules, caplets, tablets, powders, gels or other unit dosage forms).

The current invention is directed to a treatment of inflammatory diseases, for example, an autoimmune disease. Various autoimmune diseases that can be treated according to the invention include, but are not limited to, acute disseminated encephalomyelitis (ADEM), Addison's disease, Alopecia areata, Amyloidosis, Autoimmune retinopathy, autoimmune thyroid disease, Axonal and neuronal neuropathies, chronic fatigue syndrome, chronic inflammatory demyelinating polyneuropathy (CIDP), Crohn's disease, Coxsackie myocarditis, dermatitis herpetiformis, experimental allergic encephalomyelitis, Evans syndrome, Fibromyalgia, Glomerulonephritis, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Kawasaki syndrome, Lupus (SLE), Lyme disease, Meniere's disease, Multiple sclerosis, Myasthenia gravis, Myositis, Narcolepsy, Neuromyelitis optica (Devic's), Neutropenia, Scleroderma, Sjogren's syndrome, Stiff person syndrome.

In an embodiment, the inflammatory disease treated according to the invention is inflammation produced after a chemical or biological treatment, for example, chemotherapy. In one embodiment, the chemotherapy comprises administration of paclitaxel, 5-fluorouracil (5-FU), Cisplatin, Methotrexate, Actinomycin, Bleomycin, Busulfan, Capecitabine, Cyclophosphamide, Cytosine arabinoside, Daunomycin, Daunorubicin, Docetaxel, Doxil, Doxorubicin, Etoposide, Floxuridine, Hydroxyurea, Mechlorethamine, Melphalan, Mitomycin, Mitoxantrone, Procarbazine, 6-mercaptopurin, 6-thioguanine, Thiotepa, Vinblastine or Vinorelbine.

In certain embodiments, the inflammatory disease exhibits altered levels, for example, altered blood levels, of one or more cytokines or related biomolecules. In further embodiments, the inflammatory disease exhibits altered blood levels of one or more cytokines or related biomolecules selected from cluster of differentiation 40 ligand (CD-40L), Eotaxin, fibrinogen, growth hormone (GH), keratinocyte-derived cytokine or GRO1 oncogene (KC/GRO), interleukin-1β (IL-1β), IL-6, IL-18, lymphotactin, myeloperoxidase (MPO), tissue inhibitor of metalloproteinase 1 (TIMP-1), vascular endothelial growth factor A (VEGF-A), C-reactive protein (CRP), macrophage-derived chemokine (MDC), macrophage inflammatory protein-1α (MIP-1α), vWF, and oncostatin.

Table 1 provides non-limiting examples of diseases which exhibit altered levels of certain cytokines and related biomolecules.

TABLE 1
Non-limiting examples of diseases which exhibit altered levels of certain
cytokines and related biomolecules.

| Biomolecule | Inflammatory disease |
|---|---|
| CD-40L | Inflammatory bowel diseases, for example, ulcerative colitis and |
| | Crohn's disease, atherosclerosis, rheumatoid arthritis, systemic lupus |
| | erythematosus. |
| Eotaxin | Allergic airway diseases, for example, asthma and rhinitis; |
| | inflammatory bowel diseases, for example, ulcerative colitis and |
| | Chrohn's disease, gastrointestinal allergic hypersensitivity; skin- |
| | related inflammatory diseases, for example, dermatitis herpetiformis, |
| | AIDS-associated eosinophilic folliculitis, bullous pemphigoid, |
| | pemphigoid gestationis, atopic dermatitis; allergic drug reaction, T- |
| | cell mediated hepatitis, liver cirrhosis. Additional examples provided |
| | in United States Application Publication No. US20050287159, which |
| | is herein incorporated by reference in its entirety. |
| Fibrinogen | Arthritis, for example, rheumatoid arthritis, coronary artery disease, |
| | vascular wall disease, multiple sclerosis, bacterial infection, colitis, |
| | lung and kidney fibrosis, Duchenne muscular dystrophy. |
| GH | Inflammatory bowel disease, for example, ulcerative colitis and |
| | Crohn's disease, non-obese diabetes, chronic inflammatory systemic |
| | disease. |
| KC/GRO | Inflammatory bowel disease, for example, ulcerative colitis and |
| | Chrohn's disease, macrophage-mediated inflammation, atherogenesis, |
| | atherosclerosis, psoriasis. |
| IL-1β | Autoimmune conditions, for example, Muckle-Wells syndrome, |
| | chronic infantile neurologic, cutaneous and articular |
| | syndrome/neonatal onset multisystem inflammatory disease, gout, |
| | adult and juvenile Still's disease, systemic juvenile idiopathic |
| | arthritis, rheumatoid arthritis, septic shock, Cryopyrin-associated |
| | periodic syndromes (CAPS), Hyper IgD syndrome (HIDS), Schnitzler |
| | syndrome, TNF receptor-associated periodic syndrome (TRAPS), |
| | Blau syndrome, Sweet syndrome, Deficiency in IL-1 receptor |
| | antagonist (DIRA), Recurrent idiopathic pericarditis, Macrophage |
| | activation syndrome (MAS), Urticarial vasculitis, Antisynthetase |
| | syndrome, Relapsing chondritis, Behçet disease, Erdheim-Chester |
| | syndrome (histiocytosis), Synovitis, acne, pustulosis, hyperostosis, |
| | osteitis (SAPHO). Rheumatoid arthritis, Periodic fever, aphthous |
| | stomatitis, pharyngitis, adenitis syndrome (PFAPA), Type 2 diabetes, |
| | Smoldering multiple myeloma, Postmyocardial infarction heart |
| | failure, Osteoarthritis. |
| IL-6 | Rheumatoid arthritis, collagen-induced arthritis, adjuvant-induced |
| | arthritis, cardiac myxoma, polyclonal plasmacytosis, proliferative |
| | glomerulonephritis. |
| IL-8 | Inflammatory skin disease, for example, palmoplantar pustulosis, |
| | rheumatoid arthritis, inflammatory bowel disease, for example, |
| | ulcerative colitis and Crohn's disease, psoriasis, autoimmune disease |
| | involving neutrophil activation. |
| Lymphotactin | Inflammatory bowel disease, for example, ulcerative colitis and |
| | Chrohn's disease, inflammatory oral disease. |
| MPO | Acute and chronic vascular inflammatory disease, Pyoderma |
| | gangrenosum, Sweet's syndrome, T cell-mediated inflammatory |
| | diseases, dermatitis herpetoformos, systemic inflammatory response |
| | syndrome, anti-neutrophil cytoplasmic autoantibody mediated |
| | glomerulonephritis. |
| TIMP-1 | Inflammatory neurodegenerative diseases, chronic |
| | neuroinflammation, liver fibrosis, chronic hepatitis, inflammatory |
| | bowel disease, for example, ulcerative colitis and Crohn's disease, |
| | atherosclerosis, corneal ulceration |
| VEGF-A | Rheumatoid arthritis, diabetic retinopathy, inflammation induced |
| | angiogenesis, chronic inflammatory disease, inflammatory bowel |
| | disease, for example, ulcerative colitis and Crohn's disease. |
| MDC | Atopic dermatitis, lung inflammation, atherosclerosis, autoimmune |
| | encephalomyelitis. |
| MIP-1α | Bacterial sepsis, silicosis, oxidant-induced lung injury, sarcoidosis, |
| | idiopathic pulmonary fibrosis, inflammatory lung disease, CNS |
| | inflammation. |
| vWF | Inflammatory bowel disease, for example, ulcerative colitis and |
| | Crohn's disease, arterial thrombus formation, atherosclerosis. |
| oncostatin | Rheumatoid arthritis, inflammatory diseases of lung, inflammatory |
| | diseases of skin, atherosclerosis, cardiovascular disease, multiple |
| | sclerosis. |

In certain embodiments, the inflammatory disease which can be treated according to the invention involves dysregulated inflammatory mechanisms, oxidative stress and disturbance of immune homeostasis.

Conditions and disorders that can be treated with the treatment of the current invention include, but are not limited to, allergy, Alzheimer's disease, Ankylosing Spondylitis, asthma, autoimmune diseases, arthritis, atherosclerosis, Carpal Tunnel syndrome, Celiac, Crohn's disease, diverticulitis, eczema, fibrosis, Guillain-Barre Disease, lupus, multiple sclerosis, nephritis, neuropathy, pancreatitis, Parkinson's Disease, psoriasis, polymyalgia rheumatica, rheumatoid arthritis, scleroderma and vasculitis.

Non-limiting examples of autoimmune diseases which can be treated according to the invention include acute disseminated encephalomyelitis (ADEM), Addison's disease, Alopecia areata, Amyloidosis, Autoimmune retinopathy, autoimmune thyroid disease, Axonal & neuronal neuropathies, chronic fatigue syndrome, chronic inflammatory demyelinating polyneuropathy (CIDP), Crohn's disease, Coxsackie myocarditis, dermatitis herpetiformis, experimental allergic encephalomyelitis, Evans syndrome, Fibromyalgia, Glomerulonephritis, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Kawasaki syndrome, Lupus (SLE), Lyme disease, chronic, Meniere's disease, Multiple sclerosis, Myasthenia gravis, Myositis, Narcolepsy, Neuromyelitis optica (Devic's), Neutropenia, Scleroderma, Sjogren's syndrome, Stiff person syndrome.

Any of the aforementioned aspects of the invention for the treatment of an inflammatory disease may further comprise the administration of one or more additional therapy or therapies for treating an inflammatory disease. Non-limiting examples of such additional therapies include anti-inflammatory antibody therapies, for example, anti-IgE therapy, or anti-inflammatory small molecule therapies, e.g., anti-histamine therapy, steroids or non-steroidal anti-inflammatory drugs (NSAIDS) such as aspirin, celecoxib, diclofenac, diflunisal, etofolac, ibuprofen, indomethacin, ketoprofen, ketorolac, nambumetone, naproxen, oxaprozin, piroxicam, salsalate, sulindac, tolmetin or combinations thereof. Additional anti-inflammatory agents are well known to a person of ordinary skill in the art and such embodiments are within the purview of the invention.

The composition comprising ECGC, curcumin, glucosinolates and/or derivatives thereof and MCT can be administered to a subject in the form of a powder, drink, emulsion, gel, capsule, tablet or a mixture thereof. A subject in need of a treatment for an inflammatory disease may ingest the composition provided by the current invention either directly or by mixing it with other foods or drinks, for example, water, fruit juice, yogurt, soups, stews, pasta, etc. Further, the composition (or individual components) can also be incorporated into other food products, for example, cake, cookies, cereal bars, etc.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight or by calories and all solvent mixture proportions are by volume unless otherwise noted.

### Example 1—Prevention of Pro-Inflammatory Effectors in Inflammation Using LC/MCT/Curcumin/EGCG/SFN

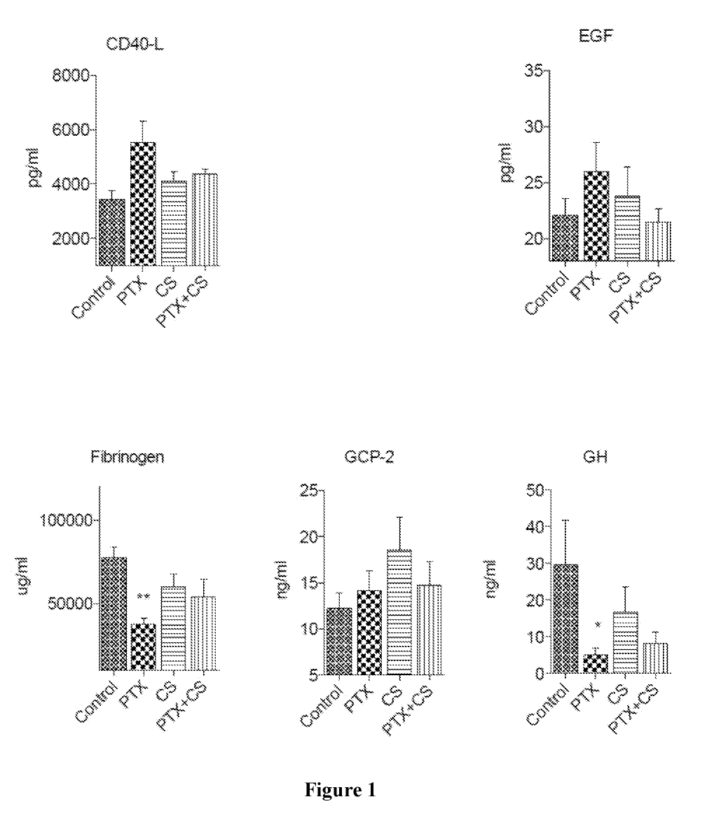

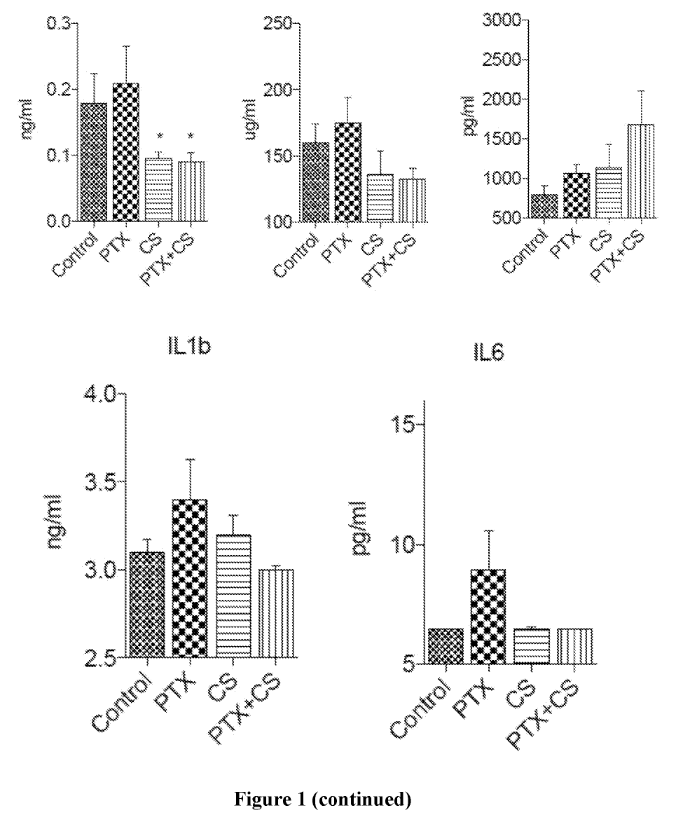

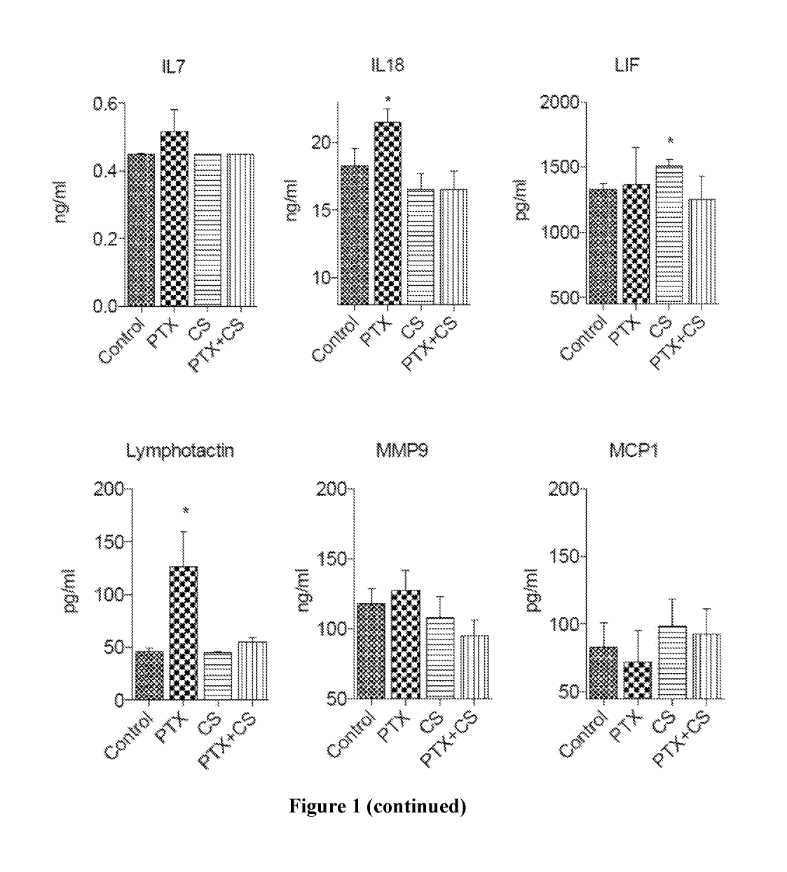

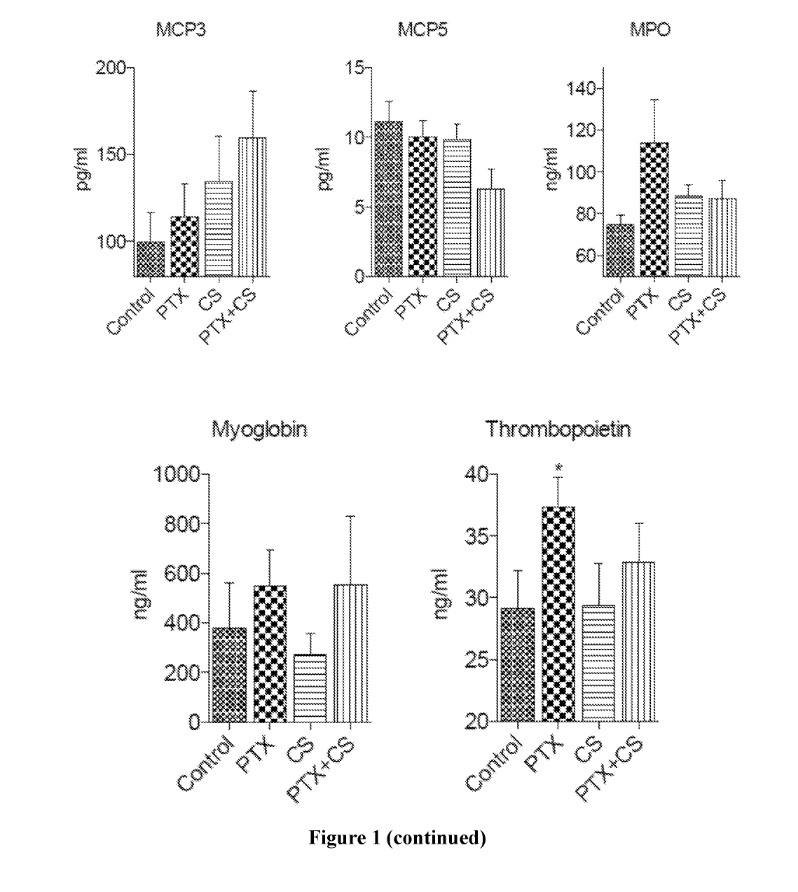

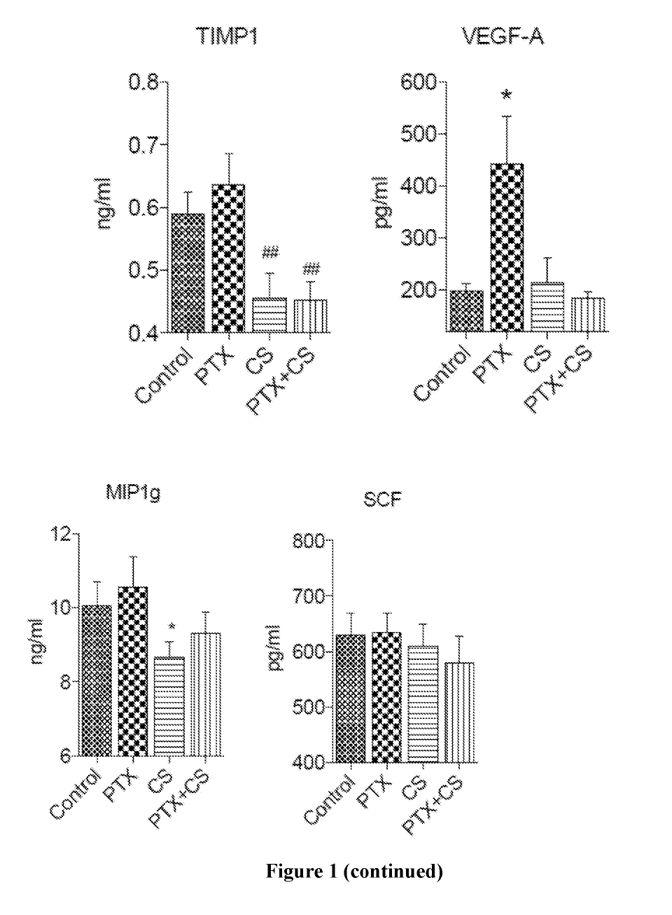

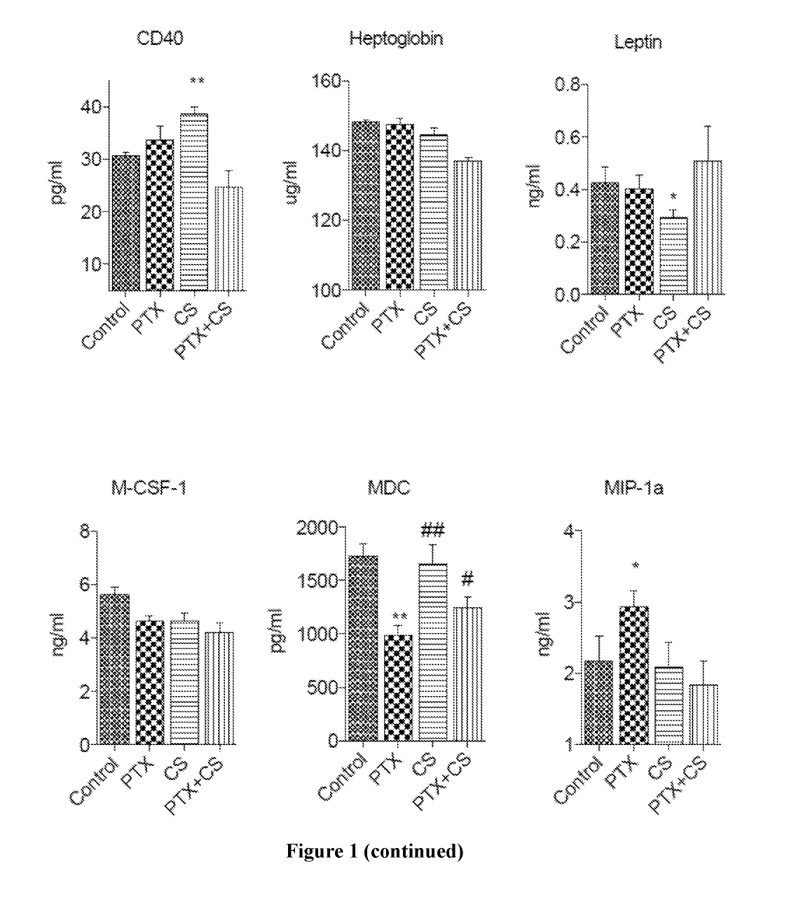

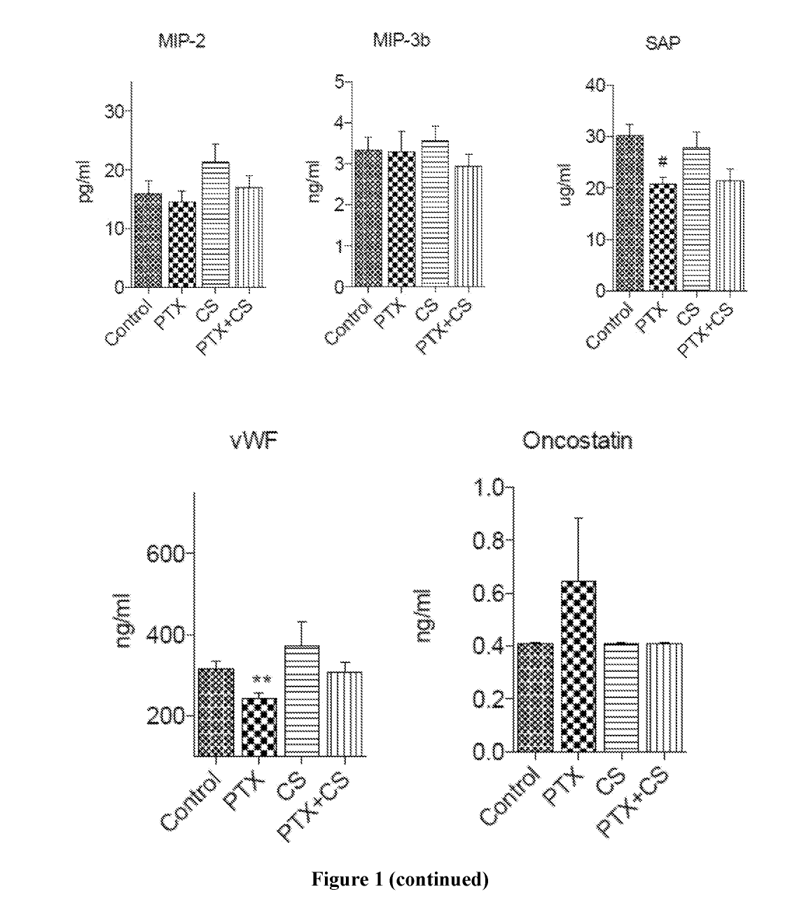

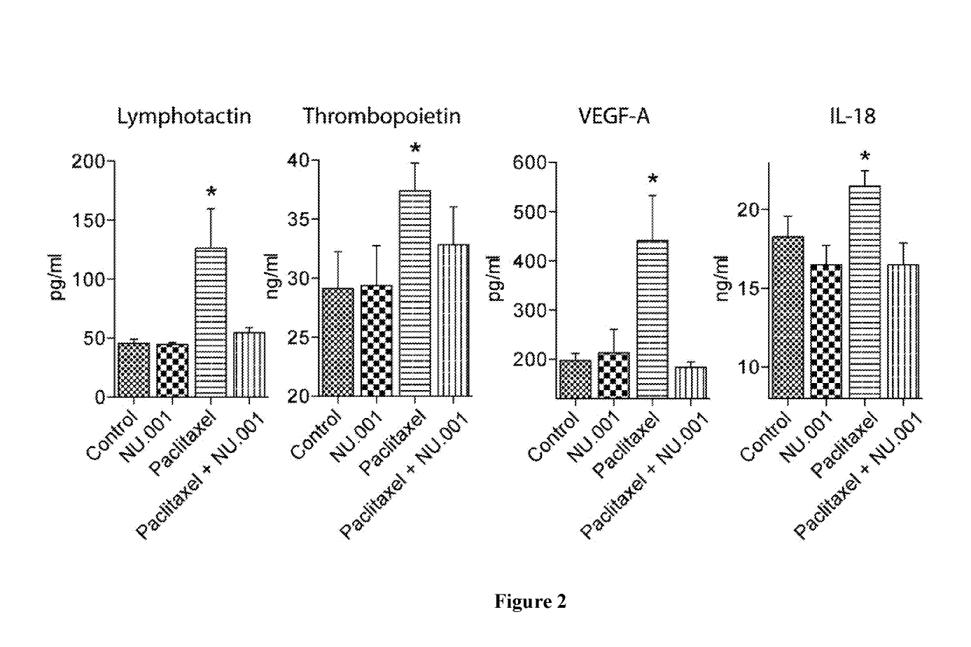

The chemotherapeutic agent paclitaxel was used to induce inflammation. Animals were stressed by treating with paclitaxel [40 mg/kg cumulative], resulting in changes in a number of cytokines and related molecules involved in inflammation processes. Animals were treated with NU.001 for 3-4 weeks and blood was taken for plasma isolation. A panel of cytokines was screened using RodentMAP [Myriad/RBM]. FIGS. 1 and 2 show the return to control levels in a number of cytokines whose expression was altered as a result of paclitaxel treatment. These results demonstrate the capacity of NU.001 to reduce inflammation.

### Example 2—NU.001 Inhibits Pro-Inflammatory Cytokines Involved in Pain and Mitogenesis

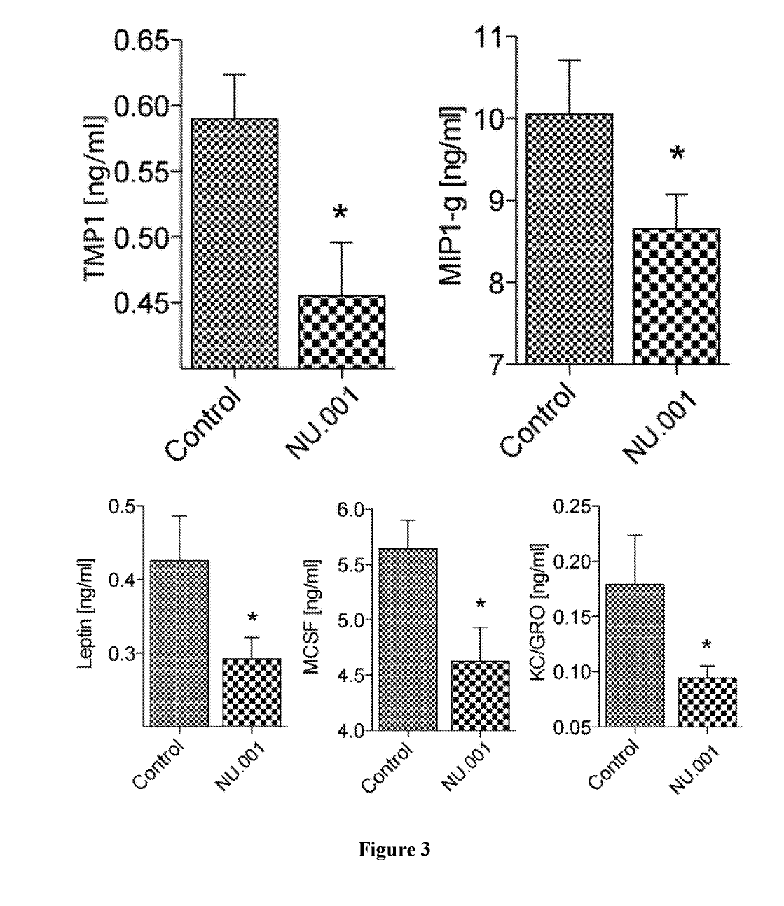

Levels of multiple pro-inflammatory cytokines were compared in animals treated for 3-4 weeks with control diet or NU.001 diets. The results of the cytokine screening revealed the ability of NU.001 to significantly inhibit TIMP-1, MIP-1 g, leptin, macrophage colony-stimulating factor (MCSF) and (KC/GRO) (FIG. 3). TIMP-1 is increased in subjects with neuropathic pain. Increased levels of MIP-1 g, leptin and M-CSF have been correlated with pain. High levels of M-CSF are also linked to ankylosing spondylitis and rheumatoid arthritis. Finally, KC/GRO has demonstrated mitogenic properties and is involved in melanoma pathogenesis. Together these data demonstrate the capabilities of NU.001 to modulate pain and mitogenesis.

### Example 3—NU.001 Stimulates Factors that Modulate Peripheral Neuropathy and Multiple Sclerosis

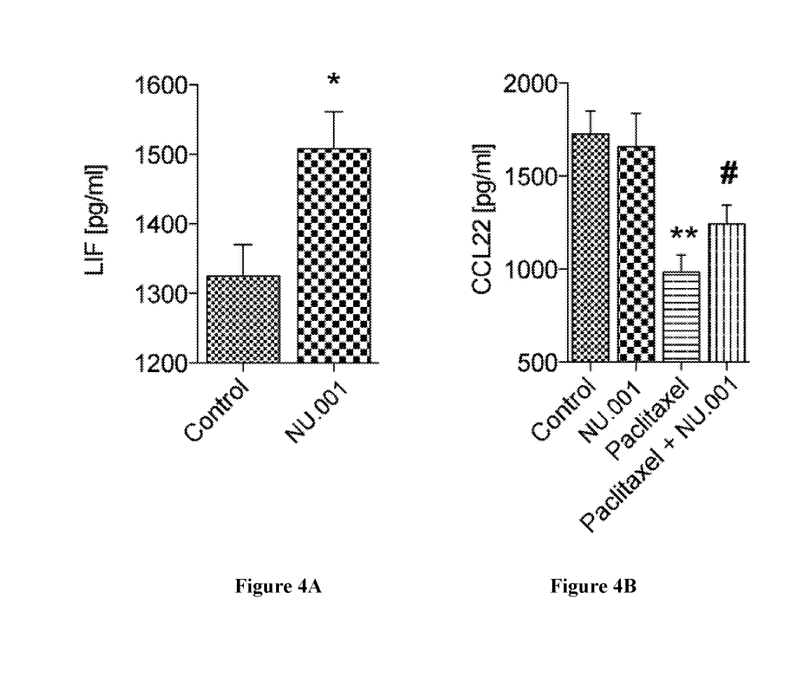

Levels of Leukemia inhibitory factor (LIF) were compared in animals treated with control diet or NU.001 diets. After 4 weeks of treatment, blood was taken and plasma was isolated to measure levels of cytokines. Results demonstrate an increased concentration of LIF in animals treated with NU.001 compared to controls, confirming the potential of our treatment to modulate neuropathy (FIG. 4A). Lower levels of CCL22, as observed in patients with multiple sclerosis, were obtained using paclitaxel. Animals treated with paclitaxel in combination with NU.001 show greater concentration of the cytokine. CCL22 is hypothesized to play a critical role in the pathogenesis of multiple sclerosis, specifically in women. These results suggest that NU.001 can be used to treat multiple sclerosis.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Youm, Y. H. et al. The ketone metabolite beta-hydroxybutyrate blocks NLRP3 inflammasome-mediated inflammatory disease. Nat Med 21, 263-269, doi:10.1038/nm.3804 (2015).
2. Yang, X. & Cheng, B. Neuroprotective and anti-inflammatory activities of ketogenic diet on MPTP-induced neurotoxicity. J Mol Neurosci 42, 145-153, doi:10.1007/s12031-010-9336-y (2010).
3. Kono, H. et al. Protective effects of medium-chain triglycerides on the liver and gut in rats administered endotoxin. Annals of surgery 237, 246-255, doi:10.1097/01.SLA.0000048450.44868.B1 (2003).
4. Bachmeier, B. E. et al. Curcumin downregulates the inflammatory cytokines CXCL1 and -2 in breast cancer cells via NFkappaB. Carcinogenesis 29, 779-789, doi:10.1093/carcin/bgm248 (2008).
5. Killian, P. H. et al. Curcumin inhibits prostate cancer metastasis in vivo by targeting the inflammatory cytokines CXCL1 and -2. Carcinogenesis 33, 2507-2519, doi:10.1093/carcin/bgs312 (2012).
6. Yun, J. M., Jialal, I. & Devaraj, S. Effects of epigallocatechin gallate on regulatory T cell number and function in obese v. lean volunteers. Br J Nutr 103, 1771-1777, doi:10.1017/5000711451000005X (2010).
7. Yang, C. S., Wang, X., Lu, G. & Picinich, S. C. Nat Rev Cancer Vol. 9, 429-439 (2009).
8. Dinkova-Kostova, A. T. & Kostov, R. V. Glucosinolates and isothiocyanates in health and disease. Trends in molecular medicine 18, 337-347, doi:10.1016/j.molmed.2012.04.003 (2012).
9. Innamorato, N. G. et al. The transcription factor Nrf2 is a therapeutic target against brain inflammation. Journal of immunology 181, 680-689 (2008).
10. Kaley, T. J. & Deangelis, L. M. Therapy of chemotherapy-induced peripheral neuropathy. Br J Haematol 145, 3-14, doi:10.1111/j.1365-2141.2008.07558.x (2009).
11. Jafarzadeh, A. et al. Lower serum levels of Th2-related chemokine CCL22 in women patients with multiple sclerosis: a comparison between patients and healthy women. Inflammation 37, 604-610, doi:10.1007/s10753-013-9775-z (2014).
12. Gasior, M., Rogawski, M. A. & Hartman, A. L. Neuroprotective and disease-modifying effects of the ketogenic diet. Behavioural Pharmacology 17, 431-439 (2006).
13. Gano, L., Patel, M. & Rho, J. M. Ketogenic Diets, Mitochondria and Neurological Diseases. Journal of lipid research, jlr.R048975, doi:10.1194/jlr.R048975 (2014).
14. Gasior, M. M., Rogawski, M. A. M. & Hartman, A. L. A. Neuroprotective and disease-modifying effects of the ketogenic diet. Behavioural Pharmacology 17, 431-439, doi:10.1097/00008877-200609000-00009 (2006).
15. Ruskin, D. N., Kawamura, J., Masahito & Masino, S. A. Reduced Pain and Inflammation in Juvenile and Adult Rats Fed a Ketogenic Diet. PLoS ONE 4, e8349, doi:10.1371/journal.pone.0008349 (2009).
16. Carl E Stafstrom, J. M. R. The Ketogenic Diet as a Treatment Paradigm for Diverse Neurological Disorders. Frontiers in Pharmacology 3, doi:10.3389/fphar.2012.00059 (2012).
17. Freeman, J. M., Kossoff, E. H. & Hartman, A. L. The ketogenic diet: one decade later. PEDIATRICS 119, 535-543, doi:10.1542/peds.2006-2447 (2007).
18. Shimazu, T. et al. Suppression of Oxidative Stress by beta-Hydroxybutyrate, an Endogenous Histone Deacetylase Inhibitor. Science, doi:10.1126/science.1227166 (2012).
19. Tipoe, G. L., Leung, T. M., Hung, M. W. & Fung, M. L. Green tea polyphenols as an anti-oxidant and anti-inflammatory agent for cardiovascular protection. Cardiovascular & hematological disorders drug targets 7, 135-144 (2007).
20. Aggarwal, B. B. & Harikumar, K. B. Potential therapeutic effects of curcumin, the anti-inflammatory agent, against neurodegenerative, cardiovascular, pulmonary, metabolic, autoimmune and neoplastic diseases. The international journal of biochemistry & cell biology 41, 40-59 (2009).
21. Nallasamy, P. et al. Sulforaphane reduces vascular inflammation in mice and prevents TNF-α-induced monocyte adhesion to primary endothelial cells through interfering with the NF-κB pathway. The Journal of nutritional biochemistry 25, 824-833, doi:10.1016/j.jnutbio.2014.03.011 (2014).
22. Yang, C., Wang, X., Lu, G. & Picinich, S. Cancer prevention by tea: animal studies, molecular mechanisms and human relevance. Nat Rev Cancer, doi:10.1038/nrc2641 (2009).
23. Wu, C. H., Wu, C. F., Huang, H. W., Jao, Y. C. & Yen, G. C. Naturally occurring flavonoids attenuate high glucose-induced expression of proinflammatory cytokines in human monocytic THP-1 cells. Mol Nutr Food Res 53, 984-995, doi:10.1002/mnfr.200800495 (2009).
24. Zheng, Y., Toborek, M. & Hennig, B. Epigallocatechin gallate-mediated protection against tumor necrosis factor-alpha-induced monocyte chemoattractant protein-1 expression is heme oxygenase-1 dependent. Metabolism: clinical and experimental 59, 1528-1535, doi:10.1016/j.metabol.2010.01.018 (2010).
25. Kim, I. B. et al. Inhibition of IL-8 production by green tea polyphenols in human nasal fibroblasts and A549 epithelial cells. Biological & pharmaceutical bulletin 29, 1120-1125 (2006).
26. Wheeler, D. S. et al. Epigallocatechin-3-gallate, a green tea-derived polyphenol, inhibits IL-1 beta-dependent proinflammatory signal transduction in cultured respiratory epithelial cells. J Nutr 134, 1039-1044 (2004).
27. Heo, J., Lee, B. R. & Koh, J. W. Protective effects of epigallocatechin gallate after UV irradiation of cultured human lens epithelial cells. Korean journal of ophthalmology: KJO 22, 183-186, doi:10.3341/kjo.2008.22.3.183 (2008).
28. Singh, R., Akhtar, N. & Haqqi, T. M. Green tea polyphenol epigallocatechin-3-gallate: inflammation and arthritis. [corrected]. Life Sci 86, 907-918, doi:10.1016/j.lfs.2010.04.013 (2010).
29. Fu, Y. & Koo, M. W. EGCG protects HT-22 cells against glutamate-induced oxidative stress. Neurotoxicity research 10, 23-30 (2006).
30. Schroeder, E. K. et al. Green tea epigallocatechin 3-gallate accumulates in mitochondria and displays a selective antiapoptotic effect against inducers of mitochondrial oxidative stress in neurons. Antioxid Redox Signal 11, 469-480, doi:10.1089/ARS.2008.2215 (2009).
31. Lee, S. J. & Lee, K. W. Protective effect of (−)-epigallocatechin gallate against advanced glycation endproducts-induced injury in neuronal cells. Biological & pharmaceutical bulletin 30, 1369-1373 (2007).
32. Mandel, S., Weinreb, O., Amit, T. & Youdim, M. B. H. Cell signaling pathways in the neuroprotective actions of the green tea polyphenol (−)-epigallocatechin-3-gallate: implications for neurodegenerative diseases. Signaling pathways in EGCG neuroprotection 88, 1555-1569, doi:10.1046/j.1471-4159.2003.02291.x (2004).
33. Mandel, S. A. et al. Multifunctional activities of green tea catechins in neuroprotection. Modulation of cell survival genes, iron-dependent oxidative stress and PKC signaling pathway. Neuro-Signals 14, 46-60, doi:10.1159/000085385 (2005).
34. Menard, C., Bastianetto, S. & Quirion, R. Neuroprotective effects of resveratrol and epigallocatechin gallate polyphenols are mediated by the activation of protein kinase C gamma. Frontiers in cellular neuroscience 7, 281, doi:10.3389/fncel.2013.00281 (2013).
35. Reznichenko, L., Amit, T., Youdim, M. B. & Mandel, S. Green tea polyphenol (−)-epigallocatechin-3-gallate induces neurorescue of long-term serum-deprived PC12 cells and promotes neurite outgrowth. J Neurochem 93, 1157-1167, doi:10.1111/j.1471-4159.2005.03085.x (2005).
36. Isbrucker, R. A., Bausch, J., Edwards, J. A. & Wolz, E. Safety studies on epigallocatechin gallate (EGCG) preparations. Part 1: genotoxicity. Food and chemical toxicology: an international journal published for the British Industrial Biological Research Association 44, 626-635, doi:10.1016/j.fct.2005.07.005 (2006).
37. Isbrucker, R. A., Edwards, J. A., Wolz, E., Davidovich, A. & Bausch, J. Safety studies on epigallocatechin gallate (EGCG) preparations. Part 2: dermal, acute and short-term toxicity studies. Food and chemical toxicology: an international journal published for the British Industrial Biological Research Association 44, 636-650, doi:10.1016/j.fct.2005.11.003 (2006).
38. Isbrucker, R. A., Edwards, J. A., Wolz, E., Davidovich, A. & Bausch, J. Safety studies on epigallocatechin gallate (EGCG) preparations. Part 3: teratogenicity and reproductive toxicity studies in rats. Food and chemical toxicology: an international journal published for the British Industrial Biological Research Association 44, 651-661, doi:10.1016/j.fct.2005.11.002 (2006).
39. Imai, K. & Nakachi, K. Cross sectional study of effects of drinking green tea on cardiovascular and liver diseases. BMJ 310, 693-696 (1995).
40. Maheshwari, R. K., Singh, A. K., Gaddipati, J. & Srimal, R. C. Multiple biological activities of curcumin: a short review. Life Sciences 78, 2081-2087, doi:10.1016/j.lfs.2005.12.007 (2006).
41. Kunnumakkara, A. B., Anand, P. & Aggarwal, B. B. Curcumin inhibits proliferation, invasion, angiogenesis and metastasis of different cancers through interaction with multiple cell signaling proteins. Cancer letters 269, 199-225, doi:10.1016/j.canlet.2008.03.009 (2008).
42. Al Moundhri, M. S., Al-Salam, S., Al Mahrouqee, A., Beegam, S. & Ali, B. H. The effect of curcumin on oxaliplatin and cisplatin neurotoxicity in rats: some behavioral, biochemical, and histopathological studies. Journal of medical toxicology: official journal of the American College of Medical Toxicology 9, 25-33, doi:10.1007/s13181-012-0239-x (2013).
43. Mendonça, L. M. et al. Curcumin reduces cisplatin-induced neurotoxicity in NGF-differentiated PC12 cells. NeuroToxicology 34, 205-211, doi:10.1016/j.neuro.2012.09.011 (2013).
44. Di Pierro, F. & Settembre, R. Safety and efficacy of an add-on therapy with curcumin phytosome and piperine and/or lipoic acid in subjects with a diagnosis of peripheral neuropathy treated with dexibuprofen. Journal of Pain Research, 497, doi:10.2147/JPR.S48432 (2013).
45. Zhao, X. et al. Curcumin exerts antinociceptive effects in a mouse model of neuropathic pain: descending monoamine system and opioid receptors are differentially involved. Neuropharmacology 62, 843-854, doi:10.1016/j.neuropharm.2011.08.050 (2012).
46. Zhao, W. C. et al. Curcumin ameliorated diabetic neuropathy partially by inhibition of NADPH oxidase mediating oxidative stress in the spinal cord. Neurosci Lett 560, 81-85, doi:10.1016/j.neulet.2013.12.019 (2014).
47. Joshi, R. P. et al. SNEDDS curcumin formulation leads to enhanced protection from pain and functional deficits associated with diabetic neuropathy: an insight into its mechanism for neuroprotection. Nanomedicine 9, 776-785, doi:10.1016/j.nano.2013.01.001 (2013).
48. Tu, X. K. et al. Curcumin Inhibits TLR2/4-NF-kappaB Signaling Pathway and Attenuates Brain Damage in Permanent Focal Cerebral Ischemia in Rats. Inflammation, doi:10.1007/s10753-014-9881-6 (2014).
49. Shah, B. H. et al. Inhibitory effect of curcumin, a food spice from turmeric, on platelet-activating factor- and arachidonic acid-mediated platelet aggregation through inhibition of thromboxane formation and Ca2+ signaling. Biochemical pharmacology 58, 1167-1172, doi:10.1016/S0006-2952(99)00206-3 (1999).
50. Shankar, T. N., Shantha, N. V., Ramesh, H. P., Murthy, I. A. & Murthy, V. S. Toxicity studies on turmeric (Curcuma longa): acute toxicity studies in rats, guineapigs &amp; monkeys. Indian journal of experimental biology 18, 73-75 (1980).
51. Deshpande, S. S., Ingle, A. D. & Maru, G. B. Chemopreventive efficacy of curcumin-free aqueous turmeric extract in 7,12-dimethylbenz[a]anthracene-induced rat mammary tumorigenesis. Cancer letters 123, 35-40, doi:10.1016/S0304-3835(97)00400-X (1998).
52. Cheng, A. L. et al. Phase I clinical trial of curcumin, a chemopreventive agent, in patients with high-risk or pre-malignant lesions. Anticancer research 21, 2895-2900 (2001).
53. Lao, C. D. et al. Dose escalation of a curcuminoid formulation. BMC Complementary and Alternative Medicine 6, 10-10, doi:10.1186/1472-6882-6-10 (2006).
54. Deodhar, S. D., Sethi, R. & Srimal, R. C. Preliminary study on antirheumatic activity of curcumin (diferuloyl methane). The Indian journal of medical research 71, 632-634 (1980).
55. Lal, B. et al. Efficacy of curcumin in the management of chronic anterior uveitis. Phytotherapy research: PTR 13, 318-322, doi:10.1002/(SICI)1099-1573(199906)13:4&lt; 318::AID-PTR445&gt; 3.0.CO; 2-7 (1999).
56. Holt, P. R. P., Katz, S. S. & Kirshoff, R. R. Curcumin therapy in inflammatory bowel disease: a pilot study. Digestive Diseases and Sciences 50, 2191-2193, doi:10.1007/s10620-005-3032-8 (2005).
57. Durgaprasad, S. S., Pai, C. G. C., Vasanthkumar, Alvres, J. F. J. & Namitha, S. S. A pilot study of the antioxidant effect of curcumin in tropical pancreatitis. The Indian journal of medical research 122, 315-318 (2005).
58. Getahun, S. M. & Chung, F. L. Conversion of glucosinolates to isothiocyanates in humans after ingestion of cooked watercress. Cancer epidemiology, biomarkers &amp; prevention: a publication of the American Association for Cancer Research, cosponsored by the American Society of Preventive Oncology 8, 447-451 (1999).
59. Hanlon, N. et al. Absolute bioavailability and dose-dependent pharmacokinetic behaviour of dietary doses of the chemopreventive isothiocyanate sulforaphane in rat. British Journal of Nutrition 99, 559-564, doi:10.1017/S0007114507824093 (2007).
60. Hanlon, N., Coldham, N., Gielbert, A. & Sauer, M. Repeated intake of broccoli does not lead to higher plasma levels of sulforaphane in human volunteers. Cancer letters (2009).
61. Fimognari, C. Sulforaphane as a promising molecule for fighting cancer. Mutation Research/Reviews in Mutation Research (2007).
62. Song, M.-Y. M. et al. Sulforaphane protects against cytokine- and streptozotocin-induced beta-cell damage by suppressing the NF-kappaB pathway. 235, 57-67, doi:10.1016/j.taap.2008.11.007 (2009).
63. Nair, S. et al. Synergistic Effects of a Combination of Dietary Factors Sulforaphane and (−) Epigallocatechin-3-gallate in HT-29 AP-1 Human Colon Carcinoma Cells. Pharmaceutical research 25, 387-399, doi:10.1007/s11095-007-9364-7 (2007).
64. Dashwood, R. H. & Ho, E. Dietary agents as histone deacetylase inhibitors: sulforaphane and structurally related isothiocyanates. Nutrition reviews 66 Suppl 1, S36-38, doi:10.1111/j.1753-4887.2008.00065.x (2008).
65. Ho, E., Clarke, J. D. & Dashwood, R. H. Dietary sulforaphane, a histone deacetylase inhibitor for cancer prevention. The Journal of nutrition 139, 2393-2396, doi:10.3945/jn.109.113332 (2009).
66. Myzak, M., Dashwood, W., Orner, G. & Ho, E. Sulforaphane inhibits histone deacetylase in vivo and suppresses tumorigenesis in Apcmin mice. The FASEB Journal (2006).
67. Mulcahy, R. T., Wartman, M. A., Bailey, H. H. & Gipp, J. J. Constitutive and beta-naphthoflavone-induced expression of the human gamma-glutamylcysteine synthetase heavy subunit gene is regulated by a distal antioxidant response element/TRE sequence. J Biol Chem 272, 7445-7454 (1997).
68. Thimmulappa, R. K. et al. Identification of Nrf2-regulated genes induced by the chemopreventive agent sulforaphane by oligonucleotide microarray. Cancer Res 62, 5196-5203 (2002).
69. Bahadoran, Z. et al. Broccoli sprouts reduce oxidative stress in type 2 diabetes: a randomized double-blind clinical trial. European Journal of Clinical Nutrition 65, 972-977, doi:10.1038/ejcn.2011.59 (2011).
70. Miller, D. M., Singh, I. N., Wang, J. A. & Hall, E. D. Administration of the Nrf2-ARE activators sulforaphane and carnosic acid attenuates 4-hydroxy-2-nonenal-induced mitochondrial dysfunction ex vivo. Free Radic Biol Med 57, 1-9, doi:10.1016/j.freeradbiomed.2012.12.011 (2013).
71. Negi, G., Kumar, A. & Sharma, S. S. Nrf2 and NF-κB modulation by sulforaphane counteracts multiple manifestations of diabetic neuropathy in rats and high glucose-induced changes. Current neurovascular research 8, 294-304 (2011).
72. Ping, Z. et al. Sulforaphane protects brains against hypoxic-ischemic injury through induction of Nrf2-dependent phase 2 enzyme. Brain Res 1343, 178-185, doi:10.1016/j.brainres.2010.04.036 (2010).
73. Myzak, M. C., Tong, P., Dashwood, W.-M., Dashwood, R. H. & Ho, E. Sulforaphane retards the growth of human PC-3 xenografts and inhibits HDAC activity in human subjects. Experimental biology and medicine (Maywood, N.J.) 232, 227-234 (2007).
74. Lakhan, S. E. & Avramut, M. Matrix metalloproteinases in neuropathic pain and migraine: friends, enemies, and therapeutic targets. Pain research and treatment 2012, 952906, doi:10.1155/2012/952906 (2012).
75. Rodriguez Parkitna, J. et al. Comparison of gene expression profiles in neuropathic and inflammatory pain. Journal of physiology and pharmacology: an official journal of the Polish Physiological Society 57, 401-414 (2006).
76. Dawes, J. M., Kiesewetter, H., Perkins, J. R., Bennett, D. L. & McMahon, S. B. Chemokine expression in peripheral tissues from the monosodium iodoacetate model of chronic joint pain. Mol Pain 9, 57, doi:10.1186/1744-8069-9-57 (2013).
77. Doupis, J. et al. Microvascular reactivity and inflammatory cytokines in painful and painless peripheral diabetic neuropathy. The Journal of clinical endocrinology and metabolism 94, 2157-2163, doi:10.1210/jc.2008-2385 (2009).
78. Bali, K. K. et al. Transcriptional mechanisms underlying sensitization of peripheral sensory neurons by granulocyte-/granulocyte-macrophage colony stimulating factors. Mol Pain 9, 48, doi:10.1186/1744-8069-9-48 (2013).
79. Yang, P. T. et al. Increased expression of macrophage colony-stimulating factor in ankylosing spondylitis and rheumatoid arthritis. Annals of the rheumatic diseases 65, 1671-1672, doi:10.1136/ard.2006.054874 (2006).
80. Anisowicz, A., Bardwell, L. & Sager, R. Constitutive overexpression of a growth-regulated gene in transformed Chinese hamster and human cells. Proc Natl Acad Sci USA 84, 7188-7192 (1987).
81. Haskill, S. et al. Identification of three related human GRO genes encoding cytokine functions. Proc Natl Acad Sci USA 87, 7732-7736 (1990).

1. A method of treating an inflammatory disease in a subject, the method comprising:
a) administering to the subject an effective amount of a composition comprising epigallocatechin-3-gallate (EGCG), curcumin, glucosinolates and/or derivatives thereof, medium chain triglycerides (MCT) or combinations thereof; and, optionally, providing a modified ketogenic diet (mKD), ketones or a ketogenic diet (KD) to the subject; or
b) providing a modified ketogenic diet or a ketogenic diet to the subject; and, optionally, administering to the subject an effective amount of a composition comprising epigallocatechin-3-gallate (EGCG), curcumin, glucosinolates and/or derivatives thereof, medium chain triglycerides (MCT) or combinations thereof, said treatment reducing the levels of one or more biomolecule by at least 40% as compared to levels of said one or more biomolecule prior to the start of said treatment.

2. The method of claim 1, wherein the derivative of glucosinolates is glucoraphanin and/or sulforaphane.

3. The method of claim 1, wherein the inflammatory disease exhibits altered levels of one or more biomolecules selected from cluster of differentiation 40 ligand (CD-40L), Eotaxin, fibrinogen, growth hormone (GH), keratinocyte-derived cytokine (KC/GRO), interleukin-1β (IL-1β), IL-6, IL-18, lymphotactin, myeloperoxidase (MPO), tissue inhibitor of metalloproteinase 1 (TIMP-1), vascular endothelial growth factor A (VEGF-A), C-reactive protein (CRP), macrophage-derived chemokine (MDC), macrophage inflammatory protein-1α (MIP-1α), vWF, and oncostatin.

4. The method of claim 1, wherein the inflammatory disease is allergy, Alzheimer's disease, Ankylosing Spondylitis, asthma, autoimmune disease, arthritis, atherosclerosis, Carpal Tunnel syndrome, Celiac, Crohn's disease, diverticulitis, eczema, fibrosis, Guillain-Barre Disease, lupus, multiple sclerosis, nephritis, neuropathy, pancreatitis, Parkinson's Disease, psoriasis, polymyalgia rheumatica, rheumatoid arthritis, scleroderma or vasculitis.

5. The method of claim 4, wherein the autoimmune disease is acute disseminated encephalomyelitis (ADEM), Addison's disease, Alopecia areata, amyloidosis, autoimmune retinopathy, autoimmune thyroid disease, axonal & neuronal neuropathies, chronic fatigue syndrome, chronic inflammatory demyelinating polyneuropathy (CIDP), Crohn's disease, Coxsackie myocarditis, dermatitis herpetiformis, experimental allergic encephalomyelitis, Evans syndrome, fibromyalgia, glomerulonephritis, Granulomatosis with Polyangiitis (GPA), Grave's disease, Guillain-Barre syndrome, Hashimoto's encephalitis, Hashimoto's thyroiditis, Hemolytic anemia, Kawasaki syndrome, Lupus, Lyme disease, Meniere's disease, multiple sclerosis, Myasthenia gravis, myositis, narcolepsy, neuromyelitis optica (Devic's), neutropenia, scleroderma, Sjogren's syndrome, or Stiff person syndrome.

6. The method of claim 1, wherein the inflammatory disease is increased inflammation caused by administration of a chemotherapeutic agent.

7. The method of claim 6, wherein the chemotherapeutic agent is paclitaxel.

8. The method of claim 1, wherein the method further comprises an additional therapy or therapies to treat the inflammatory disease.

9. The method of claim 1, wherein said method comprises providing to the subject the KD or the mKD and an effective amount of a composition comprising epigallocatechin-3-gallate, curcumin, glucosinolates and/or derivatives thereof, and medium chain triglycerides.

10. A method of treating an inflammatory disease in a subject comprising administering to the subject a combination therapy, said combination therapy comprising:
a) an effective amount of EGCG, curcumin, glucosinolates, MCT and ii) a ketogenic diet or modified ketogenic diet;
b) an effective amount of EGCG, curcumin and glucosinolates;
c) an effective amount of EGCG, curcumin, glucosinolates and MCT;
d) an effective amount of EGCG, curcumin, glucosinolates and ketones; or
e) mKD+MCT,
said treatment reducing the levels of one or more biomolecules by at least 40% as compared to levels of said one or more biomolecules prior to the start of said treatment.

11. The method of claim 10, wherein the derivative of glucosinolates is glucoraphanin and/or sulforaphane.

12. The method of claim 10, wherein the inflammatory disease exhibits altered levels of one or more biomolecules selected from cluster of differentiation 40 ligand (CD-40L), Eotaxin, fibrinogen, growth hormone (GH), keratinocyte-derived cytokine (KC/GRO), interleukin-1β (IL-1β), IL-6, IL-18, lymphotactin, myeloperoxidase (MPO), tissue inhibitor of metalloproteinase 1 (TIMP-1), vascular endothelial growth factor A (VEGF-A), C-reactive protein (CRP), macrophage-derived chemokine (MDC), macrophage inflammatory protein-1α (MIP-1α), vWF, and oncostatin.

13. The method of claim 10, wherein the inflammatory disease is allergy, Alzheimer's disease, Ankylosing Spondylitis, asthma, autoimmune disease, arthritis, atherosclerosis, Carpal Tunnel syndrome, Celiac, Crohn's disease, diverticulitis, eczema, fibrosis, Guillain-Barre Disease, lupus, multiple sclerosis, nephritis, neuropathy, pancreatitis, Parkinson's Disease, psoriasis, polymyalgia rheumatica, rheumatoid arthritis, scleroderma or vasculitis.

14. The method of claim 13, wherein the autoimmune diseases is acute disseminated encephalomyelitis (ADEM), Addison's disease, Alopecia areata, amyloidosis, autoimmune retinopathy, autoimmune thyroid disease, axonal & neuronal neuropathies, chronic fatigue syndrome, chronic inflammatory demyelinating polyneuropathy (CIDP), Crohn's disease, Coxsackie myocarditis, dermatitis herpetiformis, experimental allergic encephalomyelitis, Evans syndrome, fibromyalgia, glomerulonephritis, Granulomatosis with Polyangiitis (GPA), Grave's disease, Guillain-Barre syndrome, Hashimoto's encephalitis, Hashimoto's thyroiditis, Hemolytic anemia, Kawasaki syndrome, Lupus, Lyme disease, Meniere's disease, multiple sclerosis, Myasthenia gravis, myositis, narcolepsy, neuromyelitis optica (Devic's), neutropenia, scleroderma, Sjogren's syndrome, or Stiff person syndrome.

15. The method of claim 10, wherein the inflammatory disease is increased inflammation caused by administration of a chemotherapeutic agent.

16. The method of claim 15, wherein the chemotherapeutic agent is paclitaxel.

17. The method of claim 10, wherein the method further comprises an additional therapy or therapies to treat the inflammatory disease.

18. The method of claim 10, wherein said method comprises providing to the subject the KD or the mKD and an effective amount of a composition comprising epigallocatechin-3-gallate, curcumin, glucosinolates and/or derivatives thereof, and medium chain triglycerides.
